(19) United States
(12) Patent Application Publication
(10) Pub. No.: US 2013/0053607 A1
(43) Pub. Date: Feb. 28, 2013

(54) METHOD FOR PRODUCING A RADIOACTIVE TRACER

(71) Applicants: Céline Perollier, Rouen (FR); Kaynoush Naraghi, Mont-Saint-Aignan (FR); Sami Bayoudh, Mont-Saint-Aignan (FR)

(72) Inventors: Céline Perollier, Rouen (FR); Kaynoush Naraghi, Mont-Saint-Aignan (FR); Sami Bayoudh, Mont-Saint-Aignan (FR)

(73) Assignee: POLYINTELL, Val-De-Reuil (FR)

(21) Appl. No.: 13/583,521

(22) PCT Filed: Mar. 7, 2011
(86) PCT No.: PCT/IB2011/050954

§ 371 Date: Oct. 22, 2012

(30) Foreign Application Priority Data
Mar. 9, 2010 (FR) 1051707

(51) Int. Cl. C07C 17/093; C09K 3/00

(52) U.S. Cl. 570/141; 252/182.12

(57) ABSTRACT

A method for preparing a radioactive tracer provided with a radioactive fluorine isotope, including providing a precursor of the radioactive tracer, comprising at least one leaving group completely or partially formed by a labelling entity, wherein said leaving group can be moved by a fluoride ion, providing a molecularly imprinted polymer dedicated to the molecular recognition of at least the aforementioned labelling entity, exposing the precursor to a radioactive fluoride ion source under conditions suitable for movement of the leaving group by a radioactive fluoride ion, placing the mixture resulting from step (iii) in contact with the molecularly imprinted polymer under conditions suitable for the recognition of the labelling entity, and obtaining a solution containing the radioactive tracer provided with a radioactive isotope of fluorine obtained at the end of step (iii).

### TECHNICAL FIELD

The present invention relates to the field of molecularly imprinted polymers (MIPs).

It relates more precisely to a method for preparing a radioactive tracer provided with a radioactive isotope of fluorine in which a particular molecularly imprinted polymer is notably employed.

It also relates to a kit and the use of this kit for preparing and purifying said radioactive tracer.

### BACKGROUND

Imaging in vivo is a method that makes it possible to measure and/or visualize molecular events in vivo and in real time.

Numerous methods exist, such as NMR and ultrasound, but these methods give limited information. Two other methods based on radioactivity, namely positron emission tomography (PET) and single-photon emission computed tomography (or SPECT), can record metabolic processes in vivo and require the use of exogenous radioactive probes for visualizing a signal. These probes are specific to a tissue or to a receptor and supply a detailed image of the biological process being investigated. These two techniques are used in order to obtain a variety of information (metabolism, receptor-enzyme function, biochemical mechanisms, etc.).

More precisely, PET, which is used for investigating and visualizing human physiology by detecting radiopharmaceuticals emitting positrons, uses compounds that are radiolabeled with short-lived radioelements (¹¹C, ¹⁸F, ¹⁵O, ¹³N). It has many applications, both in diagnostics and for monitoring treatment.

Regarding PET, one problem that arises is the development of rapid synthesis techniques for introducing radioelements emitting short-lived positrons. In fact, synthesis, purification, analysis and formulation must be done in a few minutes. Generally, for a complete synthesis, a loss of 3 half-lives of the isotope in question is accepted for not losing the radioactivity.

From a practical standpoint, it is therefore necessary for the place of production of the radiopharmaceuticals to be close to their place of use.

From the standpoint of synthesis, the radioelements are generally produced by a cyclotron. The radiolabeled product supplied by the cyclotron is transferred to a hot cell, where it is converted in several steps to the final radiolabeled product. Automated systems are used in order to minimize the user's exposure to radiation. Moreover, because of the limited synthesis time due to the loss of radioactivity of each element, new technologies (microwave, microfluidic, ultrasound, SPE) have been adapted for improving the speed, reproducibility, and efficiency of the radiolabeled reactions as well as their purification. Moreover, the quantity of radioisotopes produced by the cyclotron is relatively small (of the order ranging from picomolar to nanomolar), which makes the syntheses even more difficult, making miniaturized equipment necessary. Thus, a large amount of cold reagents is used, which is favorable to the reaction kinetics.

Before a batch is released for a patient, the pharmaceutical quality of the radioactive tracer must be assured, so the latter must be characterized rapidly (HPLC, TLC, GC with MS and/or radioactivity detection), purified, formulated and sterilized.

It is also necessary to take into account the radiochemical yield (RCY) of radiosynthesis and the specific activity (SA) of the final radiopharmaceutical. The RCY is the fraction of the initial radioactivity of the sample on radiochemical separation.

¹⁸F and ¹¹C are the most suitable radioelements by virtue of their half-lives.

Regarding radiolabeling with ¹⁸F, various synthesis strategies can be envisaged and have been investigated.

It is possible for the fluorine atom to be introduced either directly in a single step (electrophilic or nucleophilic fluorination), or indirectly (a multistep synthesis may prove necessary if direct introduction of the fluorine atom causes instability of the product).

Regarding nucleophilic fluorination (which is used most), it is necessary for the reactive molecule to possess a good leaving group. This group can be a triflate, a tosylate, a mesylate, a nosylate, an iodine, chlorine or bromine atom.

New methods must then be found for facilitating purification in a minimum of time, and which can be automated.

Various methods have been mentioned:
enzyme-catalyzed fluorination (equilibrium shift)
microwaves: this method makes it possible to shorten the reaction time, reduce the amount of precursor (saving of precursor and easier purification) and increase the RCY.
microreactors
reagents supported on a solid support. Their use should facilitate purification of the final product and easy adaptation for automatic equipment. Two strategies have been developed: either the polymeric support traps the fluoride ion, or the precursor is attached to the polymeric support. Both strategies have been applied to the synthesis of [¹⁸F]FDG. However, grafting of the precursor on the resin requires many synthesis steps, which must, moreover, be optimized for each precursor.

Furthermore, other techniques employ a fluorinated purification phase.

Thus, a method is already known from WO 2010/007363 for preparing a fluorinated radioactive tracer employing a fluorinated labeling entity composed more particularly of a perfluoroalkyl chain.

The radioactive tracer is purified using a fluorinated stationary phase (Fluorous Solid Phase Extraction (FSPE)), using solvents having different fluorophilic or fluorophobic characters.

WO 2010/000409 also describes a method for preparing a fluorinated radioactive tracer employing a perfluorinated labeling entity, the purification step being performed by techniques of solid phase extraction, liquid phase extraction or by distillation.

These techniques have the drawback, however, that different operating conditions have to be used between synthesis of the radioactive tracer on the one hand, and its purification on the other hand. Moreover, the purification step involves fluorinated media which, for reasons that are obvious, are undesirable in the context of applications in the medical field.

For these reasons, there is still a need for a general method for preparing and purifying a wide range of radioactive tracers.

There is also still a need for shortening the duration of the purification step and/or for improving the quality of purification of radioactive tracers.

There is notably still a need for a method that is simple, rapid, suitable for automation and reliable, for facilitating the preparation of radioactive tracers.

The inventors discovered that it is possible to meet these needs by employing a particular method as described below.

### SUMMARY

Thus, according to a first of its aspects, the present invention relates to a method for preparing a radioactive tracer provided with a radioactive isotope of fluorine, comprising at least the steps consisting of:

i) providing a precursor of said radioactive tracer having at least one leaving group formed partly or wholly from a labeling entity, said leaving group being displaceable by a fluoride ion;

ii) providing a molecularly imprinted polymer dedicated for molecular recognition of at least said labeling entity;

iii) exposing said precursor to a radioactive fluoride ion source in conditions favorable to displacement of the leaving group by a radioactive fluoride ion;

iv) bringing the mixture resulting from step (iii) into contact with said molecularly imprinted polymer in conditions favorable to recognition of said labeling entity; and

v) recovering a solution containing the radioactive tracer provided with a radioactive isotope of fluorine obtained at the end of step (iii).

According to one embodiment, the precursor of the radioactive tracer considered according to the invention can be a compound of general formula (I):

A-L-B (I)

in which:
A represents the substrate to be functionalized with a radioactive isotope of fluorine,
L-B represents the leaving group, formed partly or wholly from a labeling entity, with L denoting a bonding function that can be displaced by a fluoride ion.

The method according to the invention is advantageous in several respects.

Firstly, the synthesis and purification of a radioactive tracer provided with a radioactive isotope of fluorine can be carried out simply, reproducibly and quickly.

The method according to the invention can therefore be useful notably for preparing radiopharmaceuticals that are suitable as agents for medical imaging, for example in positron emission tomography.

In fact, the decrease in duration of the purification step obtained owing to the method according to the invention makes it possible to prepare radiopharmaceuticals having good specific radioactivity, while maintaining a high radiochemical yield.

In contrast to the techniques already known from the prior art, the method according to the invention has the further advantage that it can be implemented in one and the same solvent medium throughout the process, i.e. from synthesis of the radioactive tracer from a precursor of said radioactive tracer, to obtaining this radioactive tracer in purified form.

In particular, the method according to the invention can be applied advantageously without addition of additional solvent media during the step of purification of the radioactive tracer.

Thus, the use of molecularly imprinted polymers makes it possible to use conventional radiofluorination solvents. In fact, a judicious choice of solvent(s) or of a mixture of solvents selected from, for example, acetonitrile and water makes it possible, advantageously, to separate, owing to the imprinted polymer, the salts from the precursor and obtain a pure fraction of radioactive tracer after radiofluorination. The method according to the invention also makes it possible to avoid using other types of solvents that are not used during radiofluorination for reasons of toxicity (the radioactive tracer may be intended for patients).

According to another of its aspects, the present invention also relates to a kit that can be used for preparing and/or purifying at least one radioactive tracer provided with a radioactive isotope of fluorine, comprising at least:

i) a precursor of said radioactive tracer having at least one leaving group formed partly or wholly from a labeling entity, said leaving group being displaceable by a fluoride ion;

ii) a molecularly imprinted polymer dedicated for molecular recognition of at least said labeling entity; and

iii) optionally, a second molecularly imprinted polymer dedicated for molecular recognition of said radioactive tracer provided with a radioactive isotope of fluorine.

According to another of its aspects, the present invention also relates to the use of a kit as defined above for purposes of preparing and purifying a radioactive tracer provided with a radioactive isotope of fluorine.

Thus, the present invention has the aim of providing users with, on the one hand, a precursor of the radioactive tracer required according to the invention, having at least one leaving group as defined above formed partly or wholly from a labeling entity and, on the other hand, a molecularly imprinted polymer dedicated for molecular recognition of said labeling entity.

According to one embodiment, users can also be provided with a compound capable of reacting with the substrate to be functionalized with a radioactive isotope of fluorine to form, after reaction with said substrate, a leaving group as defined above.

### BRIEF DESCRIPTION OF THE DRAWINGS

FIG. 1 provides a graphical representation of the HPLC results obtained from monitoring the preparation of 4-fluorobutanephenyl from the corresponding tosylates; and

FIG. 2 provides a graphical representation of the HPLC results obtained from monitoring the preparation of 4-fluorobutanephenyl from Triazole B.

### DETAILED DESCRIPTION

Method

Precursor of the Radioactive Tracer

The method according to the invention comprises at least one first step (i) consisting of providing a precursor of the radioactive tracer having at least one leaving group formed partly or wholly from a labeling entity, said leaving group being displaceable by a fluoride ion.

According to one embodiment, it can notably be a compound of general formula (I):

A-L-B (I)

in which:
A represents the substrate to be functionalized with a radioactive isotope of fluorine,
L-B represents the leaving group formed partly or wholly from a labeling entity, with L denoting a bonding function that can be displaced by a fluoride ion.

The substrate to be functionalized with a radioactive isotope of fluorine (substrate A) can be any type of substrate that is of interest in medical imaging.

It can notably be a natural or synthetic substrate selected from a metabolite, a medicinal product, a hormone, a protein, a vitamin, a receptor, a biomarker, an amino acid, a peptide, a steroid etc.

Regarding the bonding functions L that are displaceable by a fluoride ion, they form part of the general knowledge of a person skilled in the art.

As examples of leaving groups L-B suitable for the invention, we may notably mention the groups:
O—SO₂—B, the oxygen atom of O—SO₂ being bound to an sp3 or sp2 carbon atom of A;
⁺I-aryl-B, bound to an sp2 carbon atom of an aryl unit of A; and
⁺N—B, bound to an sp2 carbon atom of an aryl unit of A.

Preferably, the precursor of the radioactive tracer considered according to the invention is a compound of general formula (I), in which the leaving group L-B is O—SO₂—B, the oxygen atom of O—SO₂ being bound to an sp3 or sp2 carbon atom of A.

In the context of the present invention, the leaving group, for example a leaving group L-B as defined above, is formed partly or wholly from a labeling entity.

“Labeling entity” denotes, in the sense of the invention, any entity formed partly or wholly from at least one functional group able to interact with at least one recognition site of a molecularly imprinted polymer.

“Recognition site of a molecularly imprinted polymer” denotes, in the sense of the invention, the cavity of the matrix of the molecularly imprinted polymer that is involved effectively in recognition of a target molecule.

“Interaction”, occurring between the labeling entity and a recognition site, denotes the formation of weak bonds (for example such as van der Waals bonds, hydrogen bonds, pi donor-pi acceptor bonds, or hydrophobic interactions) and/or strong bonds (for example such as ionic bonds, covalent bonds or ionic-covalent bonds, coordination bonds and dative bonds).

According to one embodiment, the whole of the leaving group, namely L-B, can constitute the labeling entity in the sense of the invention.

According to another embodiment, only a part of the leaving group L-B, and therefore only a part of B, can constitute the labeling entity.

In this second alternative, B can be formed from said labeling entity and a spacer unit providing bonding between said entity and the bonding function L.

This spacer can be, for example, of the ethylene glycol, alkyl chain, aryl chain, etc., type.

Generally, properly speaking it does not display potential interaction with a recognition site of the associated molecularly imprinted polymer and consequently does not form part of the labeling entity in the sense of the invention.

However, not all of the labeling entities known from the prior art are suitable for application of the method according to the invention.

In fact, the labeling entity required in the context of the present invention must satisfy several requirements.

Firstly, the labeling entity must not be detrimental to execution of the fluorination reaction of the precursor of the radioactive tracer considered according to the invention.

In particular, the labeling entity must not have a labile hydrogen atom.

Thus, according to one embodiment, the labeling entity does not have acid, amine, hydroxyl, phenol and thiol functional groups.

Moreover, the labeling entity must also not be detrimental to displacement of the leaving group as defined above, by a fluoride ion during execution of the fluorination reaction of the precursor of the radioactive tracer considered according to the invention.

The choice of a labeling entity satisfying all of these conditions is within the skill set of a person skilled in the art.

According to one embodiment, the labeling entity can comprise at least one unit selected from a triazole, a pyridine, a carboxyl, an amide etc.

According to another particular embodiment, the labeling entity can be without fluorinated units, and in particular without perfluoroalkyl units, i.e. units Cₙ F₂ₙ₊₁, with n being an integer greater than or equal to 1.

Molecularly Imprinted Polymer

The method according to the invention also comprises at least one second step (ii) consisting of providing a molecularly imprinted polymer dedicated for molecular recognition of at least said labeling entity.

In other words, it is a molecularly imprinted polymer comprising at least one recognition site able to interact with at least said labeling entity.

Said molecularly imprinted polymer can be obtained by any polymerization reaction known by a person skilled in the art, and for example such as indicated below.

The step of polymerization of the MIP around a target entity employs techniques known per se by a person skilled in the art. We may thus refer to the article Peter A. G. Cormack et al., Journal of Chromatography B, 804 (2004) 173-182, which presents a review of the available techniques concerning aspects of the polymerization of MIPs.

More precisely, there are basically two possible approaches for making MIPs, the covalent approach developed by Wulff in document U.S. Pat. No. 4,127,730 and the noncovalent approach developed by Mosbach in document U.S. Pat. No. 5,110,833. These two approaches can also be combined.

It is thus possible to use the first approach of the covalent type for preparing the MIP and the second approach for obtaining recognition by noncovalent interactions, as is disclosed for example in M. J. Whitcombe et al. “A New Method for the Introduction of Recognition Site Functionality into Polymers prepared by molecular Imprinting: Synthesis and Characterization of Polymeric Receptors for Cholesterol” J. Am. Chem. Soc., 1995, 117, 7105-7111.

It is also possible to use the first and second approaches for preparing the MIP, as well as for obtaining recognition by covalent and noncovalent interactions simultaneously for one and the same target molecule. Thus, the interaction develops at least in two separate sites of the recognition site as is disclosed for example in Wulff G. et al., Macromol. Chem. Phys. 1989, 190, 1717 and 1727.

A third approach (called “semi-covalent”) consists of using, for synthesis of the MIPs, specific monomers depending on the molecule or molecules targeted, and in particular at least partly monomers derived from a target molecule, thus partly playing the role of the polymer of the matrix and partly the role of targeted entity. In other words, a proportion of these monomers, once polymerized, will be removed so as to give rise to the recognition sites.

The molecularly imprinted polymers suitable for application of the method according to the invention are preferably obtained following the noncovalent approach.

In general, these MIPs are obtained by copolymerizing monomers and crosslinking agent(s) in the presence of an entity whose imprint is required to be formed precisely. The monomers become arranged specifically around this entity, also called “target entity”, by strong or weak interactions, and then are polymerized generally in the presence of a high level of crosslinking agent. After polymerization, the entity is extracted from the polymer material and thus leaves its molecular imprint in cavities within the material, which constitute true synthetic receptors comparable to biological receptors of the antibody type.

In the context of the present invention, a chemical entity comprising at least one unit corresponding to the labeling entity as defined above is preferably used as the target entity.

The MIP or more precisely the matrix of which it is constituted can thus be formed by radical copolymerization. Vinylic monomers, monomers derived from styrene, methacrylic acid, are monomers particularly suitable for this technique. Any initiator can be used, such as azobisisobutyronitrile (AIBN).

As monomers useful for synthesis of MIPs, we may mention:
acid monomers: methacrylic acid (MAA), p-vinylbenzoic acid, acrylic acid (AA), itaconic acid, 2-(trifluoromethyl)-acrylic acid (TFMAA), acrylamido-(2-methyl)-propanesulfonic acid (AMPSA), 2-carboxyethyl acrylate,
basic monomers: 4-vinylpyridine (4-VP), 2-vinylpyridine (2-VP), 4-(5)-vinylimidazole, 1-vinylimidazole, allylamine, N,N′-diethylaminoethyl methacrylamide (DEAEM), N-(2-aminoethyl)-methacrylamide, N,N′-diethyl-4-styrylamidine, N,N,N-trimethyl aminoethyl methacrylate, N-vinylpyrrolidone (NVP), ethyl urocanate,
neutral monomers: acrylamide, methacrylamide, 2-hydroxyethyl methacrylate (2-HEMA), trans-3-(3-pyridyl)-acrylic acid, acrylonitrile (AN), methyl methacrylate (MMA), styrene, ethylstyrene.

As crosslinking agent, we may notably mention p-divinylbenzene (DVB), 1,3-diisopropenyl benzene (DIP), ethylene glycol dimethacrylate (EGDMA), tetramethylene dimethacrylate (TDMA), N,O-bisacryloyl-L-phenylalaminol, 2,6-bisacryloylamidopyridine, 1,4-phenylene diacrylamide, N,N′-1,3-phenylenebis(2-methyl-2-propenamide) (PDBMP), 3,5-bisacrylamidobenzoic acid, 1,4-diacryloyl piperazine (DAP), N,N′-methylene bisacrylamide (MDAA), N,N′-ethylene bismethacrylamide, N,N′-tetramethylene bismethacrylamide, N,N′-hexamethylene bismethacrylamide, anhydroerythritol dimethacrylate, 1,4;3,6-dianhydro-D-sorbitol-2,5-dimethacrylate, isopropoylenebis(1,4-phenylene)dimethacrylate, trimethylpropane trimethacrylate (TRIM), pentaerythritol triacrylate (PETRA), pentaerythritol tetraacrylate (PETEA).

The crosslinking agent is preferably selected from ethylene glycol dimethacrylate and divinylbenzene.

It is within the general skill set of a person skilled in the art to prepare the MIP according to the invention displaying the properties required according to the intended application, and notably the required properties of recognition for the labeling entity.

The molecular imprint can be synthesized by solution, emulsion, suspension, precipitation, or microemulsion polymerization, by dispersed phase polymerization or in conditions of preparation of microgels.

The matrix of the molecular imprint formed can be of the nature of polyacrylates, polymethacrylates, polyacrylamides, polyvinylics, polyacryleine, polyacrylonitrile, poly(vinyl alcohol), polyalkyl vinyl ketone, polybenzothiazole, bis-phenol A polycarbonate, poly(diallyl dimethylammonium chloride), polyvinyl chloride, polysiloxane, aromatic polyether, polyether sulfone, polyether imide, polyethylene imine, polyimide, polyimidazole, polyoxymethylene, polyoxazole, polyoxyphenylene, polyoxytetramethylene, polyvinyl alkyl ether, polyvinylpyrrolidone, polyvinylmethyl ketone and polysaccharides.

The MIPs according to the invention, for example obtained according to the method of preparation as described above, advantageously have predominant, or even exclusive, molecular recognition for the labeling entity.

The MIPs according to the invention have, in contrast, weaker molecular recognition for chemical entities lacking the labeling entity, for example in the form of substrate A.

This aspect is notably illustrated by example 4, which demonstrates the large difference in selectivity between these entities with respect to one and the same MIP according to the invention.

According to one embodiment, the molecularly imprinted polymer according to the invention does not have recognition sites for substrate A.

Fluorination of the Radioactive Tracer Precursor

The method according to the invention also comprises at least one third step (iii) consisting of exposing said radioactive tracer precursor as defined above to a radioactive fluoride ion source in conditions favorable to displacement of the leaving group by a radioactive fluoride ion.

During this step, a nucleophilic substitution reaction takes place on the radioactive tracer precursor at the level of its leaving group, the latter being displaced by a radioactive fluoride ion.

The reagents, solvents and operating conditions that must be used for this reaction of radiofluorination are the same as those usually employed and are well known by a person skilled in the art.

For example, cryptands such as 4,7,13,16,21,24-hexaoxa-1,10-diazabicyclo[8.8.8]-hexacosane (the compound Kryptofix® 222) or other crown ethers can be used for facilitating this reaction, and notably for intensifying the nucleophilicity of the fluorine atom in a slightly basic environment.

Preferably, the solvent medium is an aprotic polar medium.

For example, step (iii) can be carried out in an aprotic polar solvent selected from acetonitrile, dimethylformamide, tetrahydrofuran and dimethylsulfoxide.

Preferably, step (iii) is carried out in acetonitrile.

The radioactive fluoride ion source can notably be selected from potassium fluoride, cesium fluoride and a tetraalkylammonium fluoride.

Preferably, it will be potassium fluoride.

Of course, this fluorination step can be preceded by a preliminary step of protection of certain functional groups optionally present on the radioactive tracer precursor, and in particular of certain functional groups optionally present on substrate A, so that the latter do not interfere with the fluorination reaction.

The preliminary protection of these functional groups by protective groups can be effected by the usual techniques for protection of chemical groups.

At the end of this third step, a complex solution is obtained containing not only the radioactive tracer provided with a radioactive isotope of fluorine obtained following fluorination of the precursor of said radioactive tracer by the radioactive fluoride ion source, but also the unreacted precursor of said radioactive tracer in excess and the leaving groups in free form displaced during this fluorination reaction.

Step (iii) is generally carried out employing the precursor of said radioactive tracer in large excess relative to the source of fluoride ion.

In this case, the precursor of said radioactive tracer can be in the majority relative to the source of fluoride ions in the final mixture resulting from step (iii), i.e. it can represent more than 50%, notably more than 60%, for example more than 70%, of the compounds present in said mixture.

Purification of the Radioactive Tracer

The method according to the invention also comprises at least one fourth step (iv) consisting of bringing the mixture resulting from step (iii) into contact with the molecularly imprinted polymer defined above, in conditions favorable to recognition of the labeling entity, followed by a step (v) consisting of recovering a solution containing the radioactive tracer provided with a radioactive isotope of fluorine obtained at the end of step (iii).

According to one embodiment, step (iv) can be carried out in conditions favorable to recognition both of said labeling entity contained in the leaving group displaced during step (iii) and of said labeling entity present in the initial radioactive tracer precursor.

In other words, the molecularly imprinted polymer according to the invention can permit both extraction of the leaving group in free form displaced during step (iii) and of the unreacted excess radioactive tracer precursor, said extractions being performed by molecular recognition of the labeling entity.

“Extraction by molecular recognition of the labeling entity” means, in the sense of the invention, a step during which the interaction with the recognition sites of said MIP is sufficient to lead to formation of a complex composed of the MIP, provided in some or all of its recognition sites with at least the labeling entity.

Support

The MIPs according to the invention can be applied on any suitable support.

“Support” means very broadly, in the sense of the invention, any flexible or rigid solid substrate, on or in which the MIPs can be bound, bonded, deposited, synthesized in situ, filled and/or packaged.

The supports usable according to the invention can be of any nature, for example of biological, nonbiological, organic or inorganic nature or a combination thereof. They can be in any form, and notably can be in the form of particles, gels, sheets, tubes, spheres, capillaries, dots, films, wells, of any size and any shape.

For example they can be in the form of particles of uniform size, notably between 10 nm and 10 mm, preferably between 25 and 80 μm, which can then be packaged in the form of a cartridge.

In general, the MIPs can be used for example on or in a support in particular of the solid phase extraction (SPE) type, for example a cartridge, a cone, a multiwell plate, for example a 96-well plate, a patch, a tea bag, a microtube, an HPLC column, a strip, chips, slides, silica plates, thin layers, a porous surface, a nonporous surface, a microfluidic system.

According to one embodiment of the invention, the molecularly imprinted polymers can be used in an extraction column, for example in an SPE cartridge, optionally graduated.

Thus, according to one embodiment of the invention, steps (iv) and/or (vi) are carried out using said molecularly imprinted polymer in an extraction column, for example in an SPE cartridge.

A procedure for solid phase extraction generally comprises three or four steps. The first is conditioning of the adsorbent contained in the extraction cartridge, which makes it possible to wet the support by solvating the functional groups present on its surface. The second step involves percolation of the solution to be treated on the MIP, in such a way that the entities that do not have any affinity for the latter (i.e. the compounds lacking the labeling entity, such as the radioactive tracer provided with a radioactive isotope of fluorine obtained at the end of step (iii)) are not retained. Conversely, the entities displaying strong affinity for the adsorbent (namely the compounds comprising at least the labeling entity, such as the leaving group in free form displaced during step (iii) and the unreacted excess radioactive tracer precursor) remain on the support at the end of this step.

A supplementary washing step can be carried out in order to remove the entities that are weakly retained by the support (notably such as the radioactive tracer provided with a radioactive isotope of fluorine obtained at the end of step (iii)), by means of a solvent of suitable eluting strength for eluting these entities while keeping the target molecule or molecules (i.e. the compounds comprising at least the labeling entity) on the support.

At the end of this process, a solution can thus be obtained in which the proportion of entities displaying strong affinity for the adsorbent (namely the compounds comprising at least the labeling entity, such as the leaving group in free form displaced during step (iii) and the unreacted excess radioactive tracer precursor) is reduced relative to the original solution.

According to one embodiment, the method according to the invention makes it possible to recover a solution containing essentially the radioactive tracer provided with a radioactive isotope of fluorine obtained at the end of step (iii).

It can notably be a solution in which said radioactive tracer represents more than 70 mol % relative to the moles of precursor present in the initial solution.

If necessary, moreover, elution of the target molecule or molecules (i.e. the compounds comprising at least the labeling entity) can be performed by passage of a solvent specifically selected for interrupting the interactions of recognition taking place between the target molecule or molecules and the MIP while avoiding elution of the interfering entities that are strongly retained on the support, so as to release the target molecule or molecules extracted.

At the end of this extraction process, a purified solution, optionally enriched in the target molecule or molecules, can thus also be obtained.

For example, in the case of application of the method of the invention for purposes of obtaining a solution comprising predominantly, or even essentially, a radioactive tracer according to the invention, the advantage is that the target molecules of the MIP according to the invention (namely the compounds comprising at least the labeling entity, such as the leaving group in free form displaced during step (iii) and the unreacted excess radioactive tracer precursor) are retained on the support and an eluted solution is obtained comprising the radioactive tracer provided with a radioactive isotope of fluorine obtained at the end of step (iii) (retained less selectively on the support) and depleted of compounds comprising at least the labeling entity.

Typically, the solvents used during solid phase extraction can be organic solvents, for example acetonitrile, methanol, dichloromethane, aqueous solvents, for example water, buffer solutions, solvents that can be used as a mixture and with different conditions of salinity, of pH, and of polarity.

It is known, moreover, that the target molecule will be retained more specifically in an MIP if the same type of interactions develop there as those that developed during synthesis.

Thus, according to one embodiment of the invention, step (iv) can be carried out using a solvent identical or similar to the solvent used during the synthesis of said MIP.

Other types of treatment steps can be envisaged, for example solid phase micro-extraction (SPME), solid phase dynamic extraction (SPDE), stir bar sorption extraction (SBSE), capillaries, strips, chips or any miniaturized system.

According to one embodiment, step (iv) can be carried out consecutively to step (iii), and notably in the solvent medium of step (iii).

In other words, the mixture resulting from step (iii) can be used as it is for step (iv), i.e. without undergoing any preliminary treatment, and notably without undergoing modification of its solvent medium.

This aspect is illustrated notably by example 5, which describes the use of the molecularly imprinted polymer in 100% acetonitrile medium containing a fluorinated molecule (as analog of the fluorinated radioactive tracer) and the labeling entity. Simple washing with acetonitrile permits isolation of the fluorinated radioactive tracer of the labeling entity.

Additional Purification of the Radioactive Tracer

According to one embodiment, the method according to the invention can also further comprise an additional step (vi) consisting of bringing the solution resulting from step (v) into contact with a second molecularly imprinted polymer dedicated for molecular recognition of the radioactive tracer provided with a radioactive isotope of fluorine.

In other words, the method according to the invention can comprise an additional step (vi) employing a second molecularly imprinted polymer, different from the molecularly imprinted polymer defined above, said second molecularly imprinted polymer comprising at least one recognition site able to interact with at least the radioactive tracer provided with a radioactive isotope of fluorine obtained at the end of step (iii).

The second molecularly imprinted polymer can be obtained by any polymerization reaction known by a person skilled in the art, and for example by the methods described above for the first molecularly imprinted polymer, the target entity being selected this time to permit formation of recognition sites specific to the radioactive tracer, and notably of recognition sites specific to substrate A.

### EXAMPLES

### Example 1

### Synthesis of the Target Molecule: Triazole A

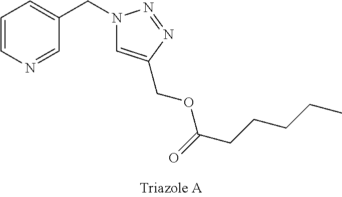 [SMILES: CCCCCC(=O)OCc1cn(Cc2cccnc2)nn1]

Propargyl Hexanoate

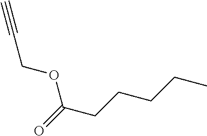

2.9 g of hexanoic acid is dissolved in 60 mL of dry THF under nitrogen. A bubbler is set up. Then 4.95 g of 1,1′-carbonyldiimidazole at 90% (1.1 eq.) is added. It is stirred at room temperature for 15 minutes. Evolution of CO₂ is observed. It is placed under nitrogen again and 1.1 equivalent of propargyl alcohol (1.62 mL) is added. It is stirred at room temperature overnight. The medium is then evaporated and then taken up in dichloromethane, it is washed with a solution of HCl at 1 mol/L and then an aqueous saturated solution of sodium chloride. The organic phase is dried over MgSO₄ and then evaporated. An oil is recovered, and is purified on a silica column. Petroleum ether and then 90/10 petroleum ether/ethyl ether are used as eluent. A colorless oil (3.1 g) is obtained at a yield of 79%.

Rf=0.79 (EdP/Et₂O 80/20), development KMnO₄.

3-Picolyl azide (Spencer et al., Organomet., 2003, 22(19), 3841).

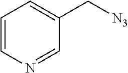

2 g of 3-picolyl chloride hydrochloride (12.2 mmol, 1 eq.) is diluted in 66 mL of water, 950 mg of sodium nitride (14.6 mmol, 1.2 eq.) is added and it is heated to 110° C. for 2 days.

The medium, once cooled, is neutralized with solid NaHCO₃ (warning: evolution of CO₂), and then the product is extracted three times with dichloromethane. The combined organic phases are dried over MgSO₄ and then evaporated in a rotary evaporator. The bath temperature is room temperature, and the product is not evaporated to dryness owing to the risk of explosion.

Rf=0.62 (DCM/EtOAc 90/10), UV detection. The initial chloride is not visible under UV.

¹H NMR (CDCl₃, 300 MHz) δ 4.32 (s, CH₂ N3, 2H), 7.25 (m, CH, 1H), 7.59 (m, CH, 1H), 8.51 (m, 2×CH, 2H).

¹³C NMR (CDCl₃, 75 MHz) δ 52.03 (CH₂ N3), 123.58 (CH), 131.11 (C^{IV}), 135.63 (CH), 149.32 (CH), 149.62 (CH).

### Triazole A

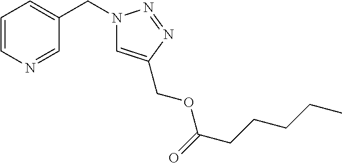 [SMILES: CCCCCC(=O)OCc1cn(Cc2cccnc2)nn1]

2 g of propargyl hexanoate (13 mmol) is diluted in a DCM/water 50/50 mixture (20 mL/20 mL). CuSO₄.5H₂O (0.05 eq., 162 mg) is added. Blue grains are observed in suspension. Sodium ascorbate (0.15 eq., 385 mg) is added, and the solution turns brown. 3-picolyl nitride diluted in dichloromethane is added dropwise. A nitrogen flask is placed on the flask. The solution becomes clear very quickly. It is stirred overnight. Then it is extracted three times with aqueous dichloromethane solution. The various organic phases are combined and dried over MgSO₄ and then evaporated under vacuum. Then purification is performed on a silica column: EdP/Et₂O 50/50 then 20/80 then DCM/MeOH 95/5. 3.9 g of triazole (beige solid) is obtained, i.e. a yield of 100%.

Rf=0.22 (DCM/EtOAc 50/50), development KMnO₄.

HPLC-MS (gradient: from 0 to 2 minutes (100% HCOOH in water), then to 18 minutes (80/20 ACN/100% HCOOH in water), then to 30 minutes (80/20 ACN/100% HCOOH in water) then return to 100% HCOOH in water): flow: 0.2 mL/min, column Hypersil Gold 50×2.1 mm, Vinj: 2 μL, tR=15.11 min, ES (+)=288.51 (MH+).

### Example 2

### Synthesis of a Precursor Bearing a Labeling Entity and Investigation of Fluorination of the Precursor Bearing a Labeling Entity (Model)

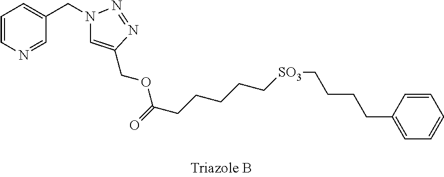

Propargyl 6-bromohexanoate

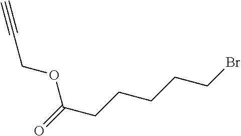

5.02 g of 6-bromohexanoic acid is dissolved in 100 mL of dry THF under nitrogen. A bubbler is set up. Then 4.64 g of 1,1′-carbonyldiimidazole (1.1 eq.) is added. It is stirred at room temperature for 15 minutes. Evolution of CO₂ is observed. It is placed under nitrogen again and 1.1 equivalent of propargyl alcohol (1.66 mL) is added. It is stirred at room temperature overnight. The medium is then evaporated and then taken up in dichloromethane, it is washed with a solution of HCl at 1 mol/L and then a saturated aqueous solution of sodium chloride. The organic phase is dried over MgSO₄ and then evaporated. An oil is recovered, and is purified on a silica column. 80/20 DCM/petroleum ether is used as eluent. A colorless oil (5.3 g) is obtained at a yield of 91%.

Rf=0.74 (DCM/EtOAc 80/20), development KMnO₄.

Propargyl 6-thiocyanatohexanoate

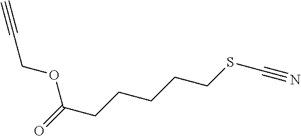

2 g of propargyl 6-bromohexanoate (8.58 mmol, 1 eq.) is dissolved in 20 mL of methanol with 925 mg of potassium thiocyanate (9.44 mmol, 1.1 eq.). It is heated to 70° C. for 20 hours. The medium is cooled, and then filtered and rinsed with methanol. The filtrate is evaporated and taken up in dichloromethane. The residue is filtered and the filtrate containing the expected product is evaporated. A silica column is used, with dichloromethane as eluent. 1.5 g of a colorless oil is recovered, i.e. a yield of 83%.

Rf=0.37 (DCM), development KMnO₄.

Rf=0.57 (DCM), development KMnO₄ for the initial product.

3-Picolyl azide triazole+propargyl 6-thiocyanatohexanoate

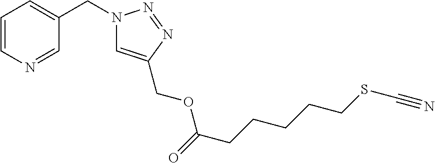 [SMILES: N#CSCCCCCC(=O)OCc1cn(Cc2cccnc2)nn1]

1.27 g of propargyl 6-thiocyanatohexanoate (6.02 mmol) is diluted in a DCM/water 50/50 mixture. CuSO₄.5H₂O (0.05 eq., 75 mg) is added. Blue grains are observed in suspension. Sodium ascorbate (0.15 eq., 179 mg) is added, and the solution turns brown. 3-picolyl nitride diluted in dichloromethane is added dropwise. A nitrogen flask is placed on the flask. The solution becomes clear very quickly. It is stirred overnight. Then it is extracted three times with aqueous dichloromethane solution. The various organic phases are combined and dried over MgSO₄ and then evaporated under vacuum. Then purification on silica is carried out. Eluent: DCM then going gradually to 100% EtOAc and then ending with DCM/MeOH 90/10 for eluting the expected product. 1.7 g of triazole (white solid) is obtained, i.e. a yield of 82%.

Rf=0.14 (EtOAc), development KMnO₄.

HPLC-MS (gradient: from 0 to 2 minutes (100% HCOOH in water), then to 18 minutes (80/20 ACN/100% HCOOH in water), then to 30 minutes (80/20 ACN/100% HCOOH in water) then return to 100% HCOOH in water): flow: 0.2 mL/min, column Hypersil Gold 50×2.1 mm, Vinj: 2 μL, tR=14.2 min, ES (+)=346.12 (MH+), 691.03 (2M+H+), ES(−)=390.13 (M+HCO₂₋).

Triazole B

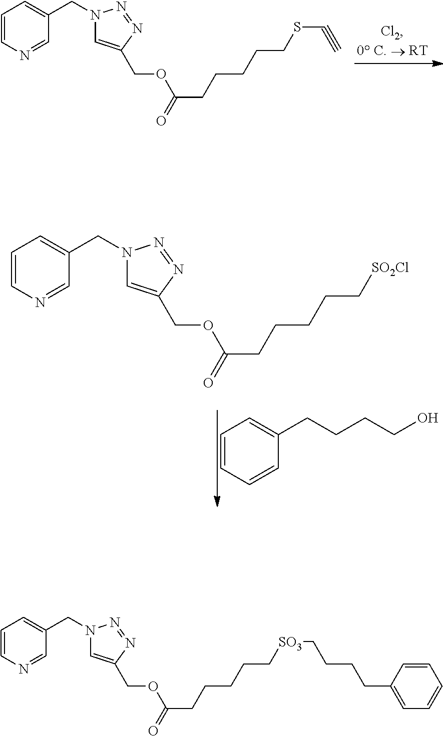

### Investigation of the Fluorination of Triazole B

Prior to fluorination of Triazole B, the compound 4-fluorobutanephenyl was prepared from the corresponding tosylate. The reaction was monitored by HPLC.

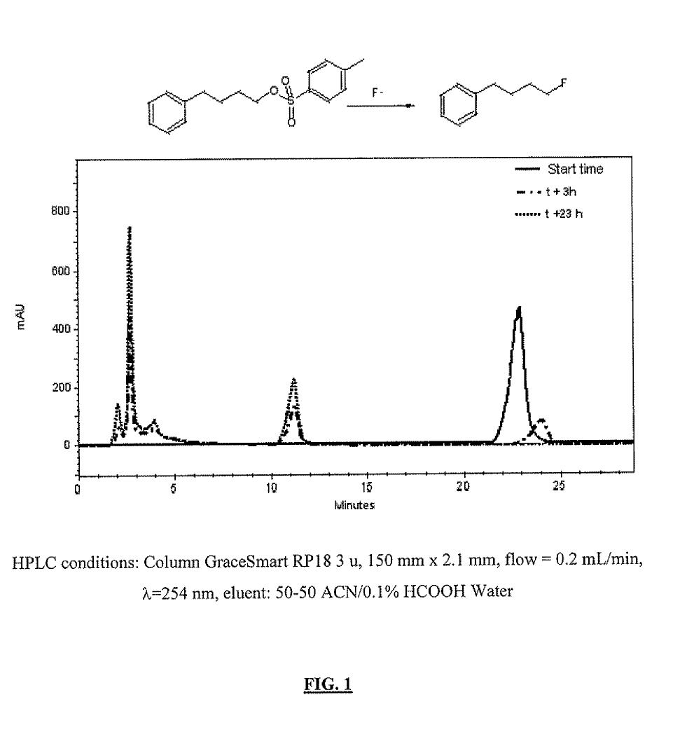

The results are presented in FIG. 1.

In the presence of tetrabutylammonium fluoride in tetrahydrofuran, disappearance of the tosylate is observed (t_{R}=22 min) to the benefit of one compound at 11 minutes and another at 3 minutes.

The product at 11 minutes is the fluorinated compound and the polar compound at 3 minutes is the residual sulfonate. The same fluorination experiment was conducted with Triazole B.

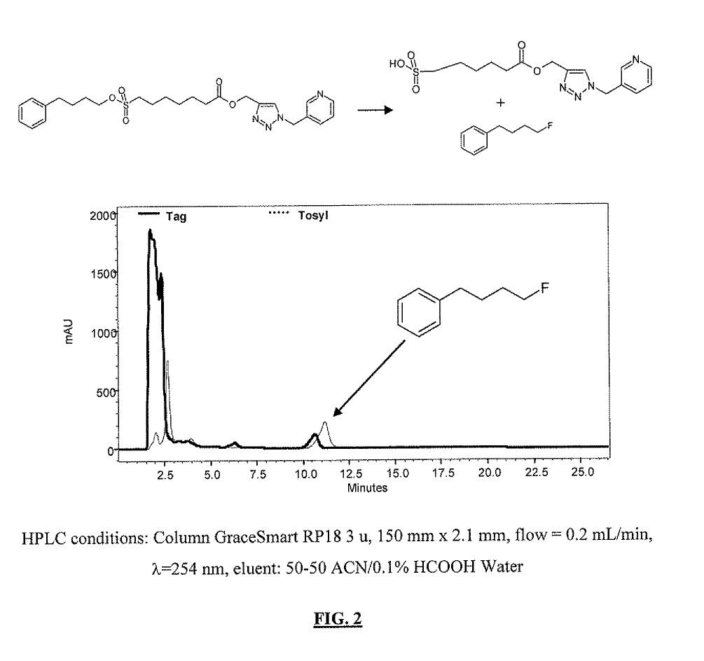

The results are presented in FIG. 2.

Using HPLC, disappearance of Triazole B is observed, and appearance of the same compound as in the preceding experiment at 11 minutes. HPLC-MS confirms the structure of Triazole B (MH+=501) as well as its disappearance after fluorination and appearance of the leaving group Tag-SO₃ H (MH−=370). Fluorination takes place on Triazole B similarly to tosylate.

Thus, the presence of the labeling entity is not detrimental to the reaction of fluorination of the precursor.

### Example 3

### Synthesis of the Corresponding Imprinted Material and Unimprinted Material No. 1 (Non-Covalent Approach)

Ethylene glycol dimethylacrylate is washed several times with a basic NaCl saturated solution to remove the inhibitor. It is dried over MgSO₄. The initiator azobisisobutyronitrile (AIBN) is recrystallized from acetone.

The imprinted material (imprint) No. 1 is prepared by mixing 299 mg of Triazole A, 4.12 g of ethylene glycol dimethylacrylate and 880 mg of methacrylic acid in 5.6 mL of anhydrous acetonitrile. The mixture is degassed by bubbling with nitrogen for minutes and then 33 mg of AIBN is added. Polymerization is carried out at 50° C. for 48 hours, forming a white monolith.

The unimprinted material No. 1 is prepared by mixing 4.12 g of ethylene glycol dimethylacrylate and 880 mg of methacrylic acid in 5.6 mL of anhydrous acetonitrile. The mixture is degassed by bubbling with nitrogen for 10 minutes and then 33 mg of AIBN is added. Polymerization is carried out at 50° C. for 48 hours, forming a white monolith.

Coarse particles (obtained from the monolith) were washed, using extraction apparatus, with a solution 5% AA MeOH—H₂O 97.5-2.5.74% of the target molecule was extracted by this washing.

After grinding and sieving between 25-45 μm, another washing of the 25-45 μm particles was carried out, using extraction apparatus with the same mixture of solvent.

Then HPLC columns of 250×2.1 mm were filled and then washed with acetonitrile at 1 mL/min to investigate recognition in HPLC.

### Example 4

### Evaluation of Recognition of Material No. 1 by HPLC

Solutions of Triazole A, Triazole B, Triazole C and cis-4-fluoro-L-proline in acetonitrile are injected on the two columns filled respectively with imprint No. 1 and with unimprinted material No. 1.

The eluent used is acetonitrile with a flow of 1 mL/min. The molecules are detected with a UV detector. The injection volumes are 20 μL.

The values of k′ (capacity factor) and of IF (imprint factor) are determined for evaluating the recognition of the various molecules on the matrixes.

| | Concentration | |
|---|---|---|
| Compound | (mg/mL) | IF |
| Triazole A | 0.566 | 2.70 |
| | 0.0566 | 3.44 |
| Triazole B | 0.29 | 2.61 |
| | 0.058 | 2.71 |
| 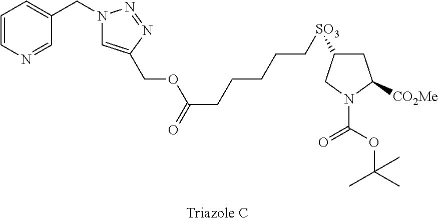 | ~0.3 ~0.03 | 2.30 2.43 |
| 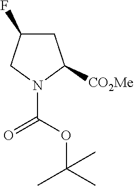 [SMILES: COC(=O)[C@@H]1C[C@H](F)CN1C(=O)OC(C)(C)C] | 5.2 2.6 | 1 1 |

HPLC conditions: eluent=ACN.

In the analysis conditions used, considerable recognition of imprint No. 1 for the various triazoles is observed.

Moreover, a significant difference is observed in recognition between the precursor provided with the labeling entity (Triazole C) and the corresponding fluorinated compound (cis-4-fluoro-L-proline).

### Example 5

### Evaluation of Recognition of Imprinted Material No. 1 by SPE

An SPE cartridge is prepared by inserting 100 mg of imprint No. 1 between two frits. Prior to extraction, 5 mL of acetonitrile is passed through the cartridge to condition it before introducing the percolating solution. Then 500 μL of a solution containing 183 μg of Triazole A and 119 μg of 4-fluorobutanephenyl in acetonitrile is percolated on the SPE cartridge. After 1.5 mL of acetonitrile has passed through, a solution is recovered containing 100% of the 4-fluorobutanephenyl with 5% of Triazole A.

The imprinted material shows selectivity for Triazole A and not for the fluorinated molecule in acetonitrile.

1. A method for producing a radioactive tracer provided with a radioactive isotope of fluorine, comprising:
i) providing a precursor of said radioactive tracer having at least one leaving group formed partly or wholly from a labeling entity, said leaving group being displaceable by a fluoride ion;
ii) providing a molecularly imprinted polymer dedicated for molecular recognition of at least said labeling entity;
iii) exposing said precursor to a radioactive fluoride ion source in conditions favorable to displacement of the leaving group by a radioactive fluoride ion;
iv) bringing the mixture resulting from step (iii) into contact with said molecularly imprinted polymer in conditions favorable to recognition of said labeling entity; and
v) recovering a solution containing the radioactive tracer provided with a radioactive isotope of fluorine obtained at the end of step (iii).

2. The method as claimed in claim 1, wherein the precursor of said radioactive tracer is a compound of general formula (I):
A-L-B (I)
in which:
A represents the substrate to be functionalized with a radioactive isotope of fluorine,
L-B represents the leaving group, formed partly or wholly from a labeling entity, with L denoting a bonding function that can be displaced by a fluoride ion.

3. The method as claimed in claim 2, wherein A represents a synthetic or natural substrate, selected from the group consisting of a metabolite, a medicinal product, a hormone, a protein, a vitamin, a receptor, a biomarker, an amino acid, a peptide and a steroid.

4. The method as claimed in claim 2, wherein the leaving group L-B is O—SO₂—B, the oxygen atom of O—SO₂ being bound to an sp3 or sp2 carbon atom of A.

5. The method as claimed in claim 2, wherein the labeling entity comprises at least one unit selected from the group consisting of a triazole, a pyridine, a carboxyl and an amide.

6. The method as claimed in claim 1, wherein the radioactive fluoride ion source is selected from the group consisting of a potassium fluoride, a cesium fluoride and a tetraalkylammonium flouride.

7. The method as claimed in claim 1, wherein step (iv) is performed in conditions favorable to recognition both of said labeling entity contained in the leaving group displaced during step (iii) and of said labeling entity present in the initial radioactive tracer precursor.

8. The method as claimed in claim 1, wherein step (iv) is performed after step (iii), and notably in the solvent medium of step (iii).

9. The method as claimed in claim 1, comprising an additional step (vi) consisting of bringing the solution resulting from step (v) into contact with a second molecularly imprinted polymer dedicated for molecular recognition of the radioactive tracer provided with a radioactive isotope of fluorine.

10. The method as claimed in claim 1, wherein steps (iv) and/or (vi) are carried out using said molecularly imprinted polymer in an extraction column, for example in an SPE cartridge.

11. A kit that can be used for preparing and/or purifying at least one radioactive tracer provided with a radioactive isotope of fluorine, comprising:
i) a precursor of said radioactive tracer having at least one leaving group formed partly or wholly from a labeling entity, said leaving group being displaceable by a fluoride ion;
ii) a molecularly imprinted polymer dedicated for molecular recognition of at least said labeling entity; and
iii) optionally a second molecularly imprinted polymer dedicated for molecular recognition of said radioactive tracer provided with a radioactive isotope of fluorine.

12. (canceled)
